(19) United States
(12) Patent Application Publication
(10) Pub. No.: US 2022/0076442 A1
(43) Pub. Date: Mar. 10, 2022

(54) DATA PROCESSING APPARATUS, IMAGE ANALYSIS METHOD, AND RECORDING MEDIUM

(71) Applicant: Preferred Networks, Inc., Tokyo (JP)

(72) Inventor: Toru MATSUOKA, Tokyo (JP)

(21) Appl. No.: 17/455,258

(22) Filed: Nov. 17, 2021

Related U.S. Application Data
(63) Continuation of application No. PCT/JP2020/019649, filed on May 18, 2020.

(30) Foreign Application Priority Data
May 21, 2019 (JP) 2019-095414

(51) Int. Cl. G06T 7/73; G06K 9/00

(52) U.S. Cl. CPC G06T 7/73; G06T 2207/20044; G06K 9/00342; G06K 9/00724

(57) ABSTRACT

A data processing apparatus includes at least one memory, and at least one processor. The at least one processor is configured to obtain, in a first coordinate system, two-dimensional data relating to a position of a predetermined part of an object in an image, calculate three-dimensional data relating to the position of the predetermined part in a second coordinate system based on the two-dimensional data relating to the position of the predetermined part in the first coordinate system, and obtain information indicating an orientation of the object based on the calculated three-dimensional data relating to the position of the predetermined part in the second coordinate system.

### CROSS-REFERENCE TO RELATED APPLICATIONS

This application is a continuation of International Application No. PCT/JP2020/019649, filed on May 18, 2020 and designating the U.S., which claims priority to Japanese Patent Application No. 2019-095414, filed on May 21, 2019. The contents of these applications are incorporated herein by reference in their entirety.

### BACKGROUND

### 1. Field of the Disclosure

The present disclosure relates to a data processing apparatus, an image distribution system, an image analysis method, and a recording medium.

### 2. Description of the Related Art

Conventionally, an image analysis technology that uses a three-dimensional camera to capture an image of a player during a game, and analyzes the orientation of the body of the player is known (see Patent Document 1, for example). For example, such an image analysis technology can accurately determine the posture of an artistic gymnastics athlete from various viewpoints.

In team sports such as soccer, there is also a need to analyze the orientation of the body (the face, the legs, or the like) of each player (hereinafter collectively referred to as “the orientation of an object”). This is because identifying the orientation of the object is useful to check player formation, and the like.

### RELATED-ART DOCUMENTS

### Patent Documents

Patent Document 1: International Publication Pamphlet No. WO2016/208290

### SUMMARY

It is desirable to provide a data processing apparatus, an image distribution system, an image analysis method, and a recording medium, in which the orientation of an object can be readily identified from obtained image data.

According to an embodiment of the present disclosure, a data processing apparatus includes at least one memory, and at least one processor. The at least one processor is configured to obtain, in a first coordinate system, two-dimensional data relating to a position of a predetermined part of an object in an image, calculate three-dimensional data relating to the position of the predetermined part in a second coordinate system based on the two-dimensional data relating to the position of the predetermined part in the first coordinate system, and obtain information indicating an orientation of the object based on the calculated three-dimensional data relating to the position of the predetermined part in the second coordinate system.

### BRIEF DESCRIPTION OF THE DRAWINGS

Other objects and further features of the present disclosure will be apparent from the following detailed description when read in conjunction with the accompanying drawings, in which:

FIG. 1 is a diagram illustrating an example of a system configuration of an image distribution system;

FIGS. 2A through 2C are first diagrams illustrating an overview of a process performed by an image analysis service providing unit;

FIGS. 3A through 3C are second diagrams illustrating the overview of the process performed by the image analysis service providing unit;

FIGS. 4A through 4C are third diagrams illustrating the overview of the process performed by the image analysis service providing unit;

FIG. 5 is a diagram illustrating an example of a hardware configuration of a data processing apparatus;

FIG. 6 is a first diagram illustrating an example of a functional configuration of the image analysis service providing unit;

FIG. 7 is a diagram illustrating details of a conversion process;

FIG. 8 is a first flowchart illustrating an image analysis service providing process;

FIG. 9 is a second diagram illustrating an example of a functional configuration of an image analysis service providing unit; and

FIG. 10 is a second flowchart illustrating a flow of an image analysis service providing process.

### DESCRIPTION OF THE EMBODIMENTS

In the following, embodiments of the present disclosure will be described with reference to the accompanying drawings. In the specification and the drawings, elements having substantially the same functions or configurations are denoted by the same reference numerals, and the description thereof will not be repeated.

### First Embodiment

### <System Configuration of Image Distribution System>

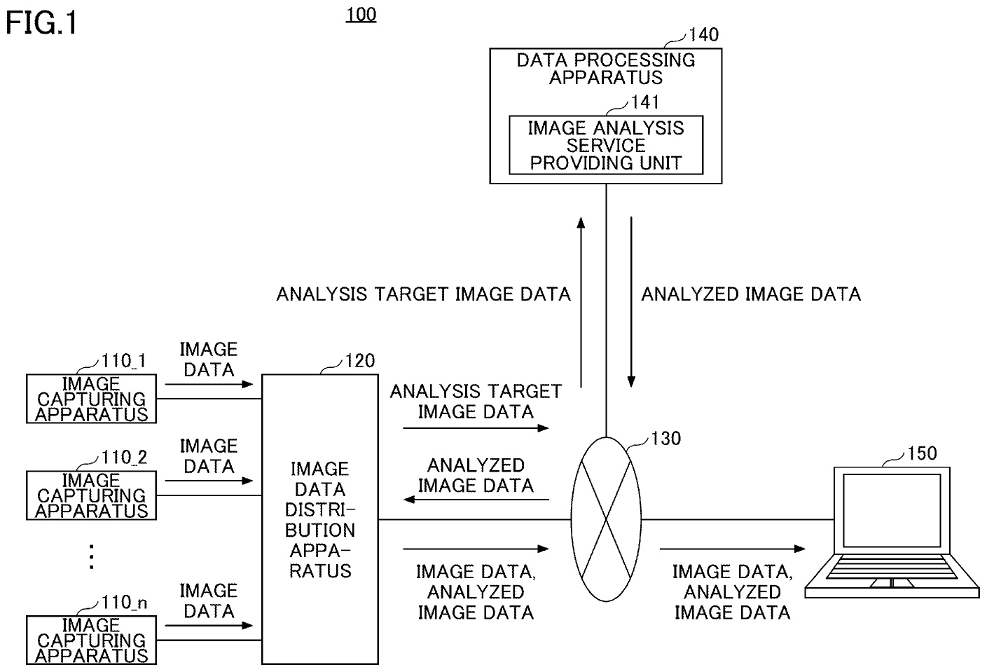

First, a system configuration of an image distribution system that includes a data processing apparatus according to a first embodiment will be described. FIG. 1 is a diagram illustrating an example of a system configuration of an image distribution system.

As illustrated in FIG. 1, an image distribution system 100 according to the present embodiment includes image capturing apparatuses 110_1 through 110_n, an image data distribution apparatus 120, a data processing apparatus 140, and a terminal 150. In the image distribution system 100, for example, the image data distribution apparatus 120, the data processing apparatus 140, and the terminal 150 are communicatively connected via a network 130.

The image capturing apparatuses 110_1 through 110_n are, for example, two-dimensional cameras configured to capture overhead images of the entire field for team sports (such as soccer, rugby, and American football). Image data obtained by the image capturing apparatuses 110_1 through 110_n are transmitted to the image data distribution apparatus 120.

It is assumed that the imaging position (three-dimensional coordinate data in the world coordinate system (a three-dimensional coordinate system)) and the imaging direction (altitude and azimuth) of each of the image capturing apparatuses 110_1 through 110_n are known fixed values. The world coordinate system is an example of a second coordinate system.

The image data distribution apparatus 120 accumulates image data transmitted from the image capturing apparatuses 110_1 through 110_n. The image data distribution apparatus 120 extracts analysis target image data from the accumulated image data, transmits the analysis target image data to the data processing apparatus 140, and requests the data processing apparatus 140 to analyze the analysis target image data. The image data distribution apparatus 120 receives the analyzed image data from the data processing apparatus 140.

Further, in response to a request from the terminal 150, the image data distribution apparatus 120 distributes the accumulated image data and the received analyzed image data to the terminal 150.

An image analysis service providing program is installed in the data processing apparatus 140, and the data processing apparatus 140 functions as an image analysis service providing unit 141 by executing the program.

The image analysis service providing unit 141 provides an image analysis service. Specifically, the image analysis service providing unit 141 receives analysis target image data, requested to be analyzed, from the image data distribution apparatus 120, and analyzes the received analysis target image data so as to generate analyzed image data. The image analysis service providing unit 141 transmits the generated analyzed image data to the image data distribution apparatus 120.

The terminal 150 is a terminal used by a user who receives image data and analyzed image data, which are distributed by the image data distribution apparatus 120. The terminal 150 may be, for example, a personal computer, a smartphone, a tablet terminal, or the like. The terminal 150 receives image data and analyzed image data from the image data distribution apparatus 120 by requesting the image data distribution apparatus 120 to distribute the image data and the analyzed image. The terminal 150 may include a display unit such as a display. Accordingly, the user of the terminal 150 can watch the image data and the analyzed image, which are distributed by the image data distribution apparatus 120.

### <Overview of Process Performed by Image Analysis Service Providing Unit>

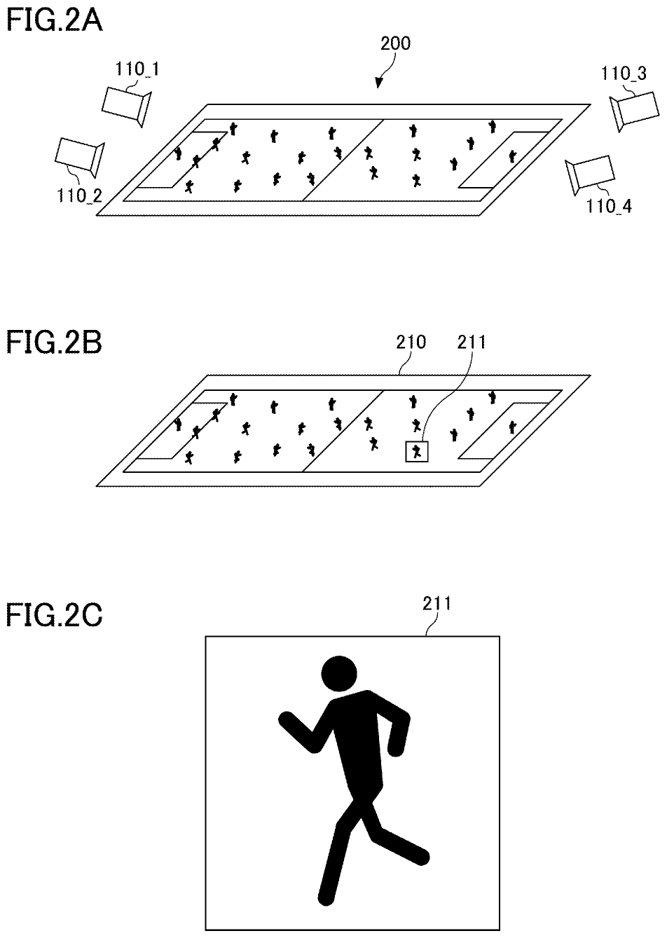

Next, an overview of a process performed by the image analysis service providing unit 141 according to the present embodiment will be described. FIGS. 2A through 4C are first through third diagrams illustrating an overview of a process performed by the image analysis service providing unit 141.

FIG. 2A depicts an example in which a plurality of image capturing apparatuses (in the example of FIG. 2A, four image capturing apparatuses 110_1 through 110_4) capture overhead images of a field 200 for soccer. The plurality of image capturing apparatuses may capture overhead images at respective imaging positions, such that each of the imaging ranges of the image capturing apparatuses includes the entire field 200 or the imaging ranges of the image capturing apparatuses include respective areas of the field 200.

FIG. 2B depicts an example of image data obtained by a predetermined image capturing apparatus (not illustrated in FIG. 2A) capturing an image of the entire field 200 from obliquely above. In the following, the image data depicted in FIG. 2B will be described as analysis target image data 210, which is to be analyzed by the image analysis service providing unit 141.

As illustrated in FIG. 2B, the image analysis service providing unit 141 identifies a region of a person in the analysis target image data 210, and extracts the region of the person as extracted image data 211. FIG. 2C depicts the enlarged extracted image data 211.

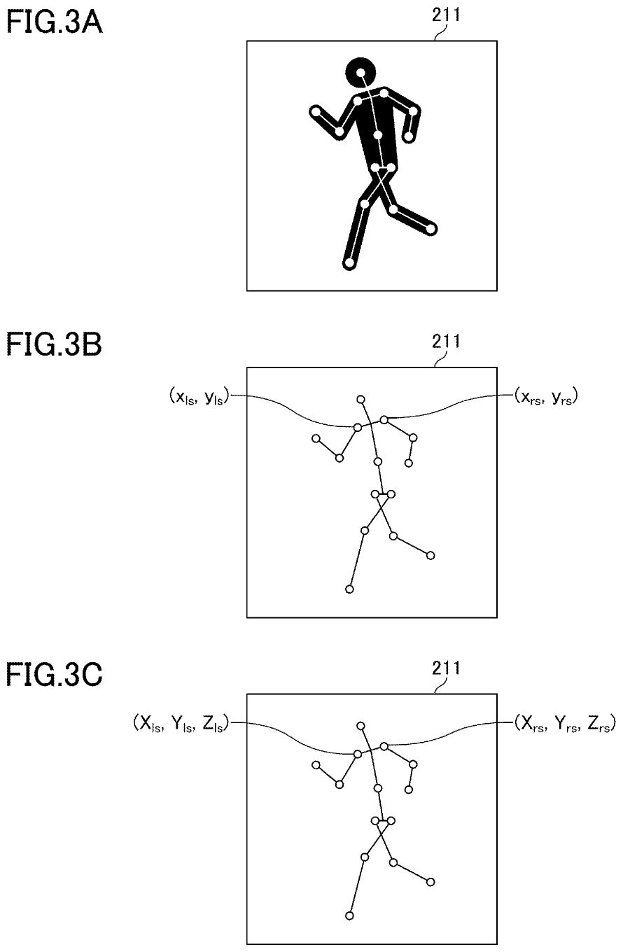

Next, the image analysis service providing unit 141 identifies the positions of feature points corresponding to parts such as the head, the shoulders, the waist, and the like of the person in the extracted image data 211. FIG. 3A depicts an example in which the positions of feature points (white dots) corresponding to parts of the person in the extracted image data 211 are identified. The positions of the feature points can be identified by, for example, image analysis. For example, the skeleton of an object can be estimated from a trained model that can use an image of the object as an input to output the skeleton of the object, and portions of the obtained skeleton can be used as feature points. In this manner, by identifying feature points based on the skeleton of an object, the image analysis service providing unit 141 can readily extract parts, such as the shoulders, the waist, and the knees, of the object having a large influence on the movement or the orientation of the object.

Next, the image analysis service providing unit 141 calculates coordinate data of the position of a feature point corresponding to the left shoulder of the person (hereinafter simply referred to as the position of the left shoulder), and coordinate data of the position of a feature point corresponding to the right shoulder of the person (hereinafter simply referred to as the position of the right shoulder), from among the identified feature points.

FIG. 3B depicts an example in which the coordinate data (xₗₛ, yₗₛ) of the position of the left shoulder of the person, and the coordinate data (xᵣₛ, yᵣₛ) of the position of the right shoulder of the person are calculated. Note that the coordinate data (two-dimensional coordinate data in an image coordinate system (a two-dimensional coordinate system)) is calculated, for example, by defining a predetermined position of the analysis target image data 210 as the origin, the horizontal direction as the x-axis, and the vertical direction as the y-axis. The image coordinate system is an example of a first coordinate system.

FIG. 3C depicts an example in which the calculated two-dimensional coordinate data is converted into three-dimensional coordinate data in the world coordinate system. Specifically, the two-dimensional coordinate data of the position of the left shoulder of the person is converted into three-dimensional coordinate data (Xₗₛ, Yₗₛ, Zₗₛ) in the world coordinate system. In addition, the two-dimensional coordinate data of the position of the right shoulder of the person is converted into three-dimensional coordinate data (Xᵣₛ, Yᵣₛ, Zᵣₛ) in the world coordinate system.

Note that the image analysis service providing unit 141 uses the imaging position and the imaging direction, in the world coordinate system, of the image capturing apparatus that has captured the image data 210 to convert the two-dimensional coordinate data in the image data 210 into the three-dimensional coordinate data in the world coordinate system. The conversion process will be described later in detail.

Next, the image analysis service providing unit 141 derives a vector representing the orientation of the body of the person (more specifically, the orientation of the chest of the person), based on the three-dimensional coordinate data of the position of the left shoulder of the person and the three-dimensional coordinate data of the position of the right shoulder of the person.

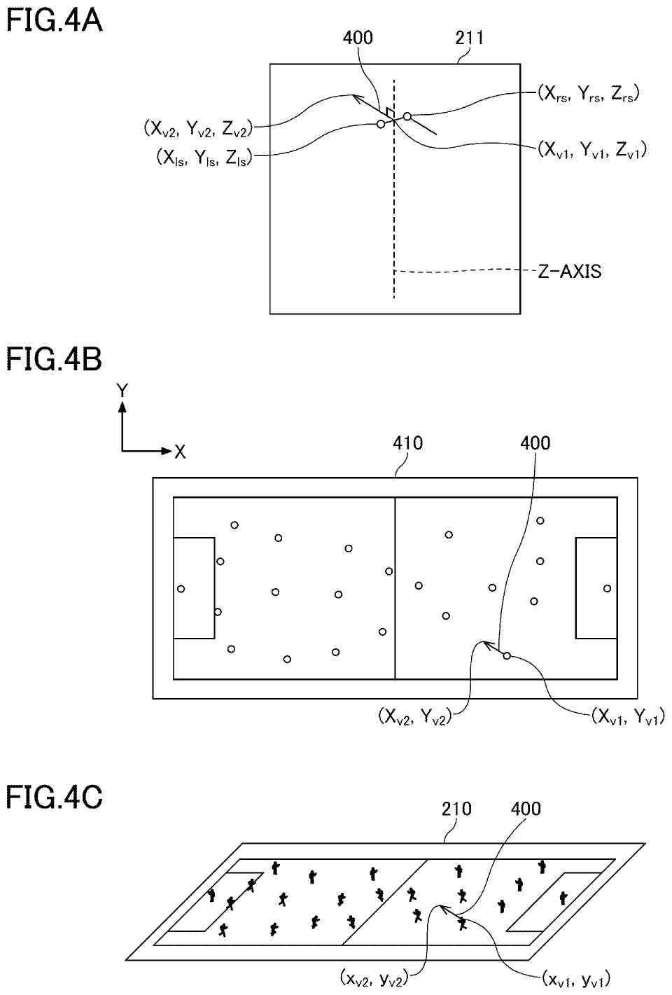

FIG. 4A depicts an example in which the vector representing the orientation of the body of the person is derived. As illustrated in FIG. 4A, according to the present embodiment, the image analysis service providing unit 141 derives a vector 400 in the world coordinate system.
The vector 400 is perpendicular to a plane that includes the position of the left shoulder of the person and the position of the right shoulder of the person, and that is parallel to the Z-axis direction.
The initial point of the vector 400 is at the midpoint between the position of the left shoulder of the person and the position of the right shoulder of the person.
The terminal point of the vector 400 is on the front side of the person.
The vector 400 has a predetermined length. In the example of FIG. 4A, the three-dimensional coordinate data ((Xₗₛ, Yₗₛ, Zₗₛ) and (Xᵣₛ, Yᵣₛ, Zᵣₛ)) of the shoulders of the person is used to derive three-dimensional coordinate data (Xᵥ₁, Yᵥ₁, Zᵥ₁) of the initial point of the vector 400 and three-dimensional coordinate data (Xᵥ₂, Yᵥ₂, Zᵥ₂) of the terminal point of the vector 400.

Next, the image analysis service providing unit 141 projects the vector 400 onto image data (in this example, a top view image viewed from above the field 200, hereinafter referred to as “projection image data”) obtained by capturing a predetermined area and associated with the world coordinates.

FIG. 4B depicts an example in which the vector 400 is projected onto projection image data 410, which is a top view image viewed from above the field 200 and in which pixels are associated with the world coordinates.

As illustrated in FIG. 4B, the vector 400 is projected onto the projection image data 410 at positions specified by coordinate data (Xᵥ₁, Yᵥ₁) and (Xᵥ₂, Yᵥ₂). The coordinates data (Xᵥ₁, Yᵥ₁) and (Xᵥ₂, Yᵥ₂) are obtained by excluding the z coordinates from the three-dimensional coordinate data of the initial point and the terminal point of the vector 400. Accordingly, the image analysis service providing unit 141 can specify the vector representing the orientation of the body of the person in the projection image data 410, which is viewed from a viewpoint different from that of the analysis target image data 210.

In the example of FIG. 4B, the vector 400 is projected onto the projection image data 410 viewed from a viewpoint different from that of the analysis target image data 210; however, the vector 400 may be projected onto the analysis target image data 210.

In this case, an inverse conversion process of the conversion process for converting two-dimensional coordinate data into three-dimensional coordinate data as described in FIG. 3C is performed for the three-dimensional coordinate data of each of the initial point and the terminal point of the vector 400.

FIG. 4C depicts an example in which two-dimensional coordinate data (xᵥ₁, yᵥ₁) and (xᵥ₂, yᵥ₂) is calculated by performing the inverse conversion process for the three-dimensional coordinate data of each of the initial point and the terminal point of the vector 400, and the vector 400 is projected onto the analysis target image data 210. Accordingly, the image analysis service providing unit 141 can specify the vector representing the orientation of the body of the person, not only in the projection image data 410 viewed from a viewpoint different from that of the analysis target image data 210, but also in the analysis target image data 210 itself.

Note that the vector 400 is not necessarily projected onto the projection image data 410 or the analysis target image data 210, and may be utilized for other purposes. For example, the vector 400 may be used as training data of a machine learning model.

### <Hardware Configuration of Data Processing Apparatus>

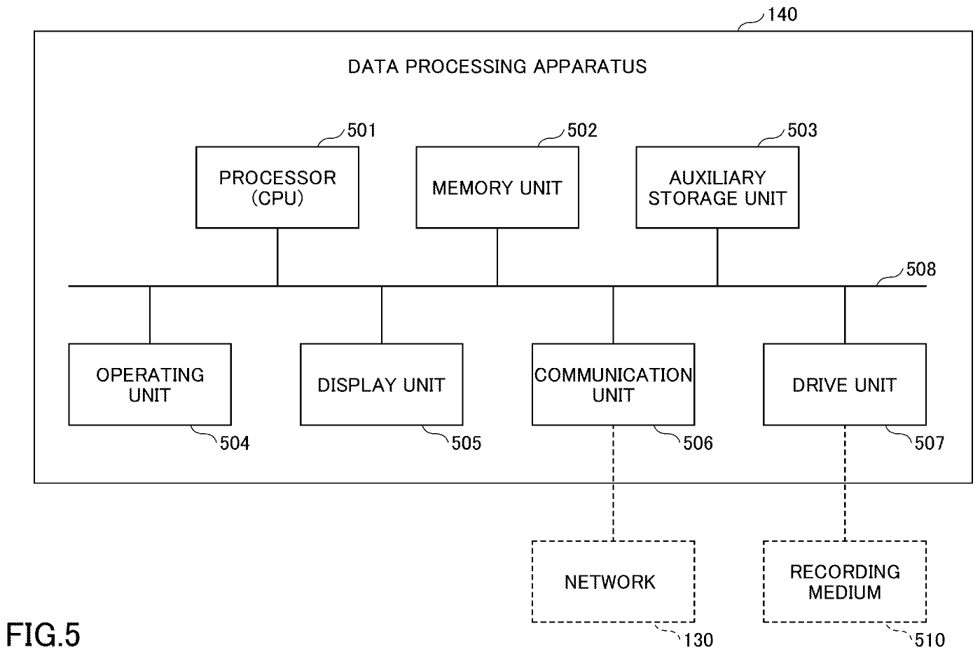

Next, a hardware configuration of the data processing apparatus 140 according to the present embodiment will be described. FIG. 5 is a diagram illustrating an example of a hardware configuration of the data processing apparatus. As illustrated in FIG. 5, the data processing apparatus 140 according to the present embodiment includes a processor 501, a memory unit 502, an auxiliary storage unit 503, an operating unit 504, a display unit 505, a communication unit 506, and a drive unit 507. The data processing apparatus 140 is implemented, for example, as a computer including these components, which are connected via a bus 508.

In the example of FIG. 5, the number of each of the components included in the data processing apparatus 140 is one; however, the number of each of the components may be plural. Further, in the example of FIG. 5, the one data processing apparatus 140 is depicted; however, a plurality of data processing apparatuses may be employed. In this case, software (such as the image analysis service providing program) may be installed in the plurality of data processing apparatuses, and the plurality of data processing apparatuses may be configured to execute different processes of the software. Further, the data processing apparatuses may communicate with each other via a network interface, or the like. Further, the image capturing apparatuses 110_1 through 110_n, the image data distribution apparatus 120, the terminal 150, or the like may perform some of processes performed by the data processing apparatus 140.

The processor 501 is an electronic circuit (such as a processing circuit or processing circuitry) including an arithmetic device such as a central processing unit (CPU). The processor 501 performs an arithmetic process based on data and a program input from the components included in the data processing apparatus 140, and outputs an arithmetic result or a control signal to the components. Specifically, the processor 501 controls the components included in the data processing apparatus 140 by executing an operating system (OS), an application, or the like.

The processor 501 is not particularly limited to a specific processing circuit as long as processes as described above can be performed. As used herein, the processing circuit may refer to one or more electronic circuits disposed on one chip or may refer to one or more electronic circuits disposed on two or more chips or two or more devices. If multiple electronic circuits are used, the electronic circuits may communicate with each other in a wired manner or in a wireless manner.

The memory unit 502 is a storage device that stores electronic information such as commands and data executed by the processor 501. The electronic information stored in the memory unit 502 is read by the processor 501. The auxiliary storage unit 503 is a storage device other than the memory unit 502. These storage devices may be any electronic components that can store electronic information, and may be memories or storage devices. The memories may be either volatile memories or non-volatile memories. A memory for storing electronic information in the data processing apparatus 140 may be implemented by the memory unit 502 or the auxiliary storage unit 503.

The operating unit 504 is an input device with which the administrator of the data processing apparatus 140 inputs various types of instructions for the data processing apparatus 140. The display unit 505 is a display device that displays analyzed image data obtained by the processor 501 executing the image analysis service providing program.

The communication unit 506 is a communication device for connecting to the network 130 and communicating with the image data distribution apparatus 120.

The drive unit 507 is a device in which a recording medium 510 is set. As used herein, the recording medium 510 includes a medium that records information optically, electrically, or magnetically, such as a CD-ROM, a flexible disk, a magneto-optical disk, or the like. The recording medium 510 may include a semiconductor memory that electrically records information, such as a ROM, a flash memory, or the like.

Various programs to be installed in the auxiliary storage unit 503 are installed, for example, when the recording medium 510 is set in the drive unit 507 and various programs recorded in the recording medium 510 are read by the drive unit 507. Alternatively, various programs to be installed in the auxiliary storage unit 503 may be installed by downloading through the network 130.

### <Functional Configuration of Image Analysis Service Providing Unit>

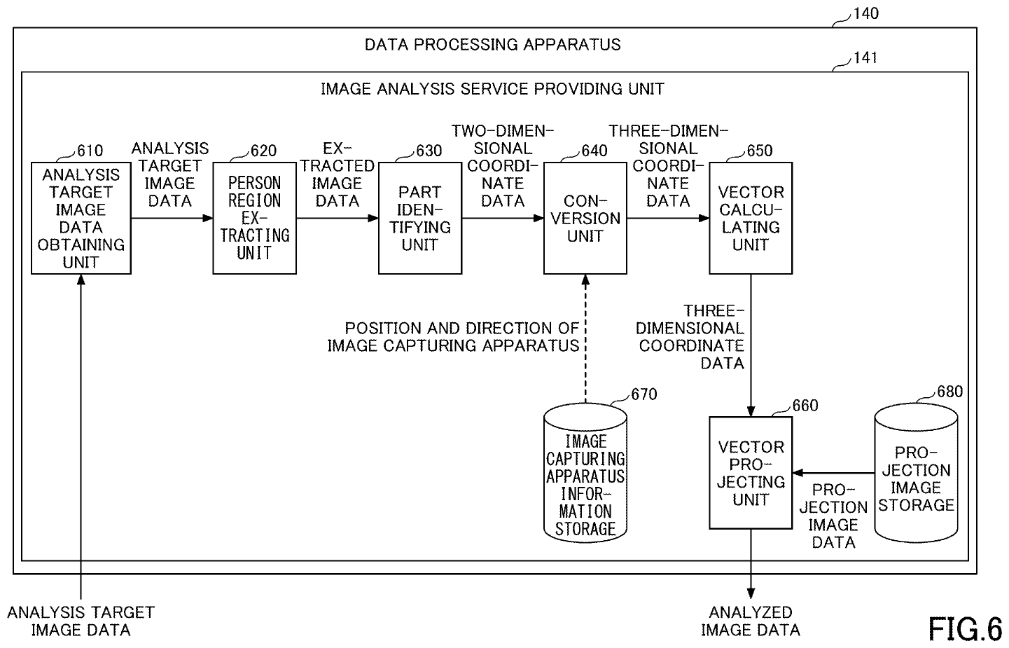

Next, a functional configuration of the image analysis service providing unit 141 according to the present embodiment will be described in detail. FIG. 6 is a diagram illustrating an example of a functional configuration of the image analysis service providing unit 141. As illustrated in FIG. 6, the image analysis service providing unit 141 according to the present embodiment includes an analysis target image data obtaining unit 610, a person region extracting unit 620, a part identifying unit 630, a conversion unit 640, a vector calculating unit 650, and a vector projecting unit 660.

The analysis target image data obtaining unit 610 obtains analysis target image data (such as the analysis target image data 210), which is requested to be analyzed, from the image data distribution apparatus 120. The analysis target image data obtaining unit 610 provides the obtained analysis target image data to the person region extracting unit 620.

The person region extracting unit 620 identifies a region of a person in the analysis target image data, and extracts image data (for example, the extracted image data 211) that includes the region of the person. The person region extracting unit 620 provides the extracted image data to the part identifying unit 630.

The part identifying unit 630 is an example of an identifying unit, and identifies the positions of feature points corresponding to respective parts of the person in the extracted image data. From among the identified feature points, the part identifying unit 630 calculates two-dimensional coordinate data of the position of a feature point corresponding to the left shoulder (the position of the left shoulder) of the person and two-dimensional coordinate data of the position of a feature point corresponding to the right shoulder (the position of the right shoulder) of the person. Further, the part identifying unit 630 provides the calculated two-dimensional coordinate data to the conversion unit 640.

The conversion unit 640 is an example of a first conversion unit, and reads the imaging position and the imaging direction of an image capturing apparatus, which has captured the analysis target image data, in the world coordinate system from an image capturing apparatus information storage 670. Further, the conversion unit 640 uses the read imaging position and the imaging direction of the image capturing apparatus to convert the two-dimensional coordinate data of the position of the left shoulder of the person and the two-dimensional coordinate data of the position of the right shoulder of the person into three-dimensional coordinate data of the position of the left shoulder of the person and three-dimensional coordinate data of the position of the right shoulder of the person in the world coordinate system.

Further, the conversion unit 640 provides the three-dimensional coordinate data of the position of the left shoulder of the person and the three-dimensional coordinate data of the position of the right shoulder of the person to the vector calculating unit 650.

The vector calculating unit 650 derives a vector (three-dimensional coordinate data of an initial point and a terminal point in the world coordinate system) representing the orientation of the body of the person, based on the three-dimensional coordinate data of the position of the left shoulder of the person and the three-dimensional coordinate data of the position of the right shoulder of the person. Further, the vector calculating unit 650 provides the derived vector (for example, the vector 400) to the vector projecting unit 660.

The vector projecting unit 660 is an example of an obtaining unit, and obtains information indicating the orientation of an object viewed from a predetermined viewpoint (in this example, the vector representing the orientation of the person). Specifically, the vector projecting unit 660 reads projection image data (for example, the projection image data 410), which is stored in a projection image storage 680 in advance, and projects the vector onto the projection image data. Further, the vector projecting unit 660 transmits the projection image data, onto which the vector has been projected, to the image data distribution apparatus 120 as analyzed image data.

### <Description of Conversion Process Performed by Conversion Unit>

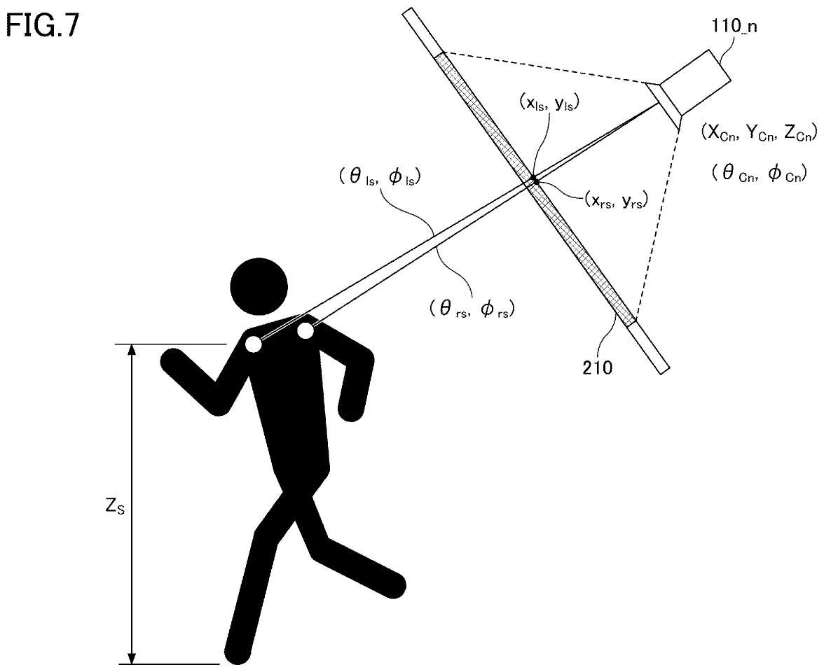

Next, the “conversion process” in which the conversion unit 640 converts two-dimensional coordinate data in analysis target image data into three-dimensional coordinate data in the world coordinate system will be described in detail. FIG. 7 is a diagram illustrating details of the conversion process.

As illustrated in FIG. 7, coordinate data (three-dimensional coordinate data in the world coordinate system) of the imaging position of an image capturing apparatus 110_n is assumed to be (X_{Cn}, Y_{Cn}, Z_{Cn}). Further, the imaging direction (altitude and azimuth) of the image capturing apparatus 110_n is assumed to be (θ_{Cn}, φ_{Cn}).

The part identifying unit 630 calculates two-dimensional coordinate data (xₗₛ, yₗₛ) of the position of the left shoulder of a person in the analysis target image data 210, which is obtained by capturing an overhead image under certain imaging conditions. As described above, the direction (altitude and azimuth) of the center position of the analysis target image data 210 viewed from the imaging position (X_{Cn}, Y_{Cn}, Z_{Cn}) is (θ_{Cn}, φ_{Cn}). Therefore, the conversion unit 640 can calculate the direction (altitude and azimuth) (θₗₛ, φₗₛ) of the position of the left shoulder of the person based on the amount of deviation between the center position and the two-dimensional coordinate data of the position of the left shoulder of the person.

Similarly, the part identifying unit 630 calculates two-dimensional coordinate data (xᵣₛ, yᵣₛ) of the position of the right shoulder of the person in the analysis target image data 210, which is obtained by capturing the overhead image under the imaging conditions. The conversion unit 640 can calculate the direction (altitude and azimuth) (ers, φᵣₛ) of the position of the right shoulder of the person based on the amount of deviation between the center position and the two-dimensional coordinate data of the position of the right shoulder of the person.

Because the heights of the left and right shoulders of the person are approximately the same, it is assumed that the Z coordinate of each of the positions of the left and right shoulders in the world coordinate system is “Zₛ” (in this example, Zₗₛ=Zᵣₛ=Zₛ, assuming that the Z coordinate of the ground of the field 200 in the world coordinate system is “0”). Under the above assumption, the conversion unit 640 calculates the point of intersection of a line, extending from the imaging position (X_{Cn}, Y_{Cn}, Z_{Cn}) of the image capturing apparatus 110_n toward the direction (θₗₛ, φₗₛ) of the position of the left shoulder of the person, and a plane that is parallel to the XY-plane with the Z-coordinate being Zₛ. Accordingly, the conversion unit 640 can calculate the X coordinate (xₗₛ) and the Y coordinate (yₗₛ) of the position of the left shoulder of the person in the world coordinate system.

Similarly, the conversion unit 640 calculates the point of intersection of a line, extending from the imaging position (X_{Cn}, Y_{Cn}, Z_{Cn}) of the image capturing apparatus 110_n toward the direction of the position of the right shoulder (θᵣₛ, φᵣₛ) of the person, and a plane that is parallel to the XY-plane with the Z-coordinate being Zₛ. Accordingly, the conversion unit 640 can calculate the X coordinate (xᵣₛ) and the Y coordinate (yᵣₛ) of the position of the right shoulder of the person in the world coordinate system.

As described above, the conversion unit 640 can convert two-dimensional coordinate data in analysis target image data into three-dimensional coordinate data (create three-dimensional coordinate data) in the world coordinate system in a simple manner. As a result, the vector projecting unit 660 can use the three-dimensional coordinate data to apply a vector representing the orientation of the body of a person to an image viewed from any viewpoint. That is, the vector projecting unit 660 can readily specify a vector, representing the orientation of the body of a person, in projection image data viewed from any viewpoint.

### <Flow of Image Analysis Service Providing Process>

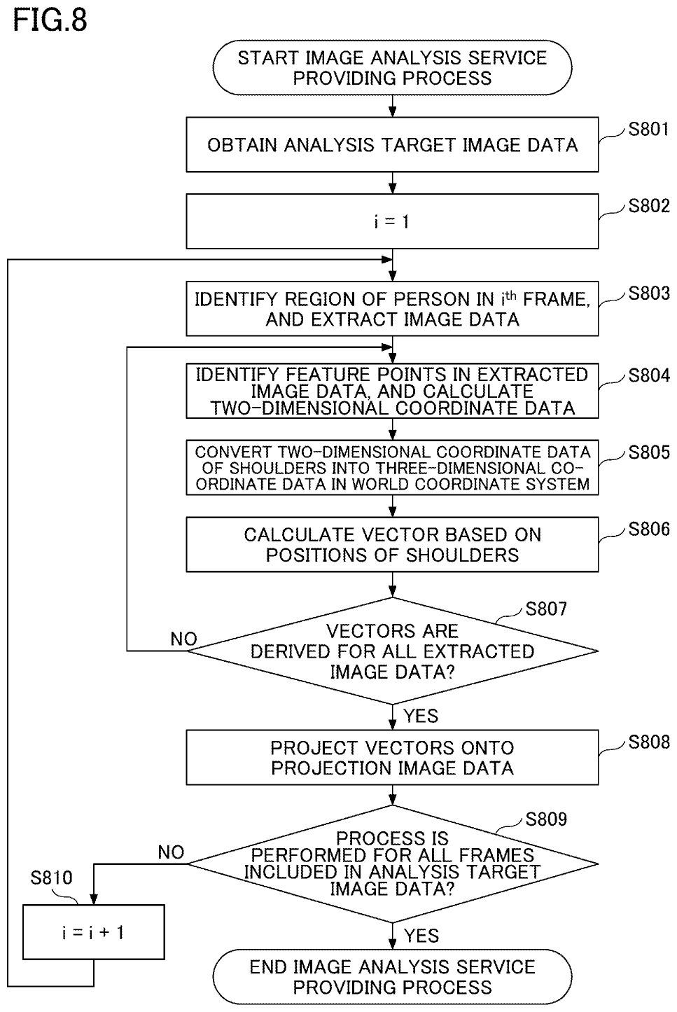

Next, a flow of an image analysis service providing process performed by the image analysis service providing unit 141 of the data processing apparatus 140 will be described. FIG. 8 is a first flowchart illustrating a flow of an image analysis service providing process.

In step S801, the analysis target image data obtaining unit 610 obtains analysis target image data.

In step S802, the person region extracting unit 620 sets a counter i to 1. The counter i counts the number of frames included in the analysis target image data.

In step S803, the person region extracting unit 620 identifies a region of a person in an i^{th} frame, and extracts, from the i^{th} frame, image data that includes the identified region of the person.

In step S804, the part identifying unit 630 identifies the positions of feature points corresponding to respective parts of the person in the extracted image data, and calculates two-dimensional coordinate data of the positions of the left shoulder and the right shoulder.

In step S805, the conversion unit 640 converts the two-dimensional coordinate data of the positions of the left shoulder and the right shoulder into three-dimensional coordinate data in the world coordinate system.

In step S806, the vector calculating unit 650 uses the three-dimensional coordinate data of the positions of the left shoulder and the right shoulder to derive a vector representing the orientation of the body of the person (a vector derived based on the positions of the shoulders).

In step S807, the person region extracting unit 620 determines whether vectors are derived for all image data extracted from the i^{th} frame. If the person region extracting unit 620 determines that there is image data for which a vector has not been derived based on the positions of shoulders (no in step S807), the process returns to step S804.

Conversely, in step S807, if the person region extracting unit 620 determines that vectors have been derived for all image data extracted from the i^{th} frame (yes in step S807), the process proceeds to step S808.

In step S808, the vector projecting unit 660 projects all the derived vectors onto projection image data. Further, the vector projecting unit 660 transmits the projection image data, onto which the vectors have been projected, to the image data distribution apparatus 120 as analyzed image data.

In step S809, the person region extracting unit 620 determines whether the process has been performed for all the frames included in the obtained analysis target image data. If the person region extracting unit 620 determines that there is a frame for which the process has not been performed (no in step S809), the process proceeds to step S810.

In step S810, the person region extracting unit 620 increments the counter i, and the process returns to step S803.

Conversely, in step S809, if the person region extracting unit 620 determines that the process has been performed for all the frames included in the analysis target image data (yes in step S809), the image analysis service providing process ends.

### SUMMARY

As is clear from the above description, the data processing apparatus according to the first embodiment is configured to:
identify the positions of the left shoulder and the right shoulder of a person in obtained analysis target image data;
convert two-dimensional coordinate data of the positions of the left shoulder and the right shoulder of the person in the analysis target image data into three-dimensional coordinate data in the world coordinate system, based on the position and the direction where the analysis target image data is obtained and also based on the heights of the left shoulder and the right shoulder of the person;
calculate a vector representing the orientation of the body of the person based on the three-dimensional coordinate data of the positions of the left shoulder and the right shoulder of the person; and
obtain a vector from a predetermined viewpoint based on the three-dimensional coordinate data of the positions of the left shoulder and the right shoulder of the person.

Accordingly, in the first embodiment, two-dimensional coordinate data of the position of a feature point corresponding to a predetermined part of a person in analysis target image data (in an image) can be converted into three-dimensional coordinate data. As a result, a vector representing the orientation of the body of the person can be derived based on the position of the feature point, and can be specified in projection image data viewed from any viewpoint.

That is, according to the first embodiment, the data processing apparatus, the image distribution system, the image analysis method, and the recording medium, in which the orientation of an object can be readily identified from obtained image data, is provided.

### Second Embodiment

In the above-described first embodiment, the positions of the right shoulder and the left shoulder of a person are identified in order to derive a vector representing the orientation of the body of the person. However, if a person is moving at a speed greater than or equal to a predetermined speed, a vector representing the orientation of the body of the person can be derived based on the direction of movement. This is because if the person is moving (for example, if the person is running) at a speed greater than or equal to a predetermined speed, the direction of movement of the person coincides with the orientation of the body of the person.

Accordingly, in the second embodiment, if a person is moving at a speed greater than or equal to a predetermined speed, a vector representing the orientation of the body of the person can be derived based on the direction of movement. In the second embodiment, differences from the above-described first embodiment will be described.

### <Functional Configuration of Image Analysis Service Providing Unit>

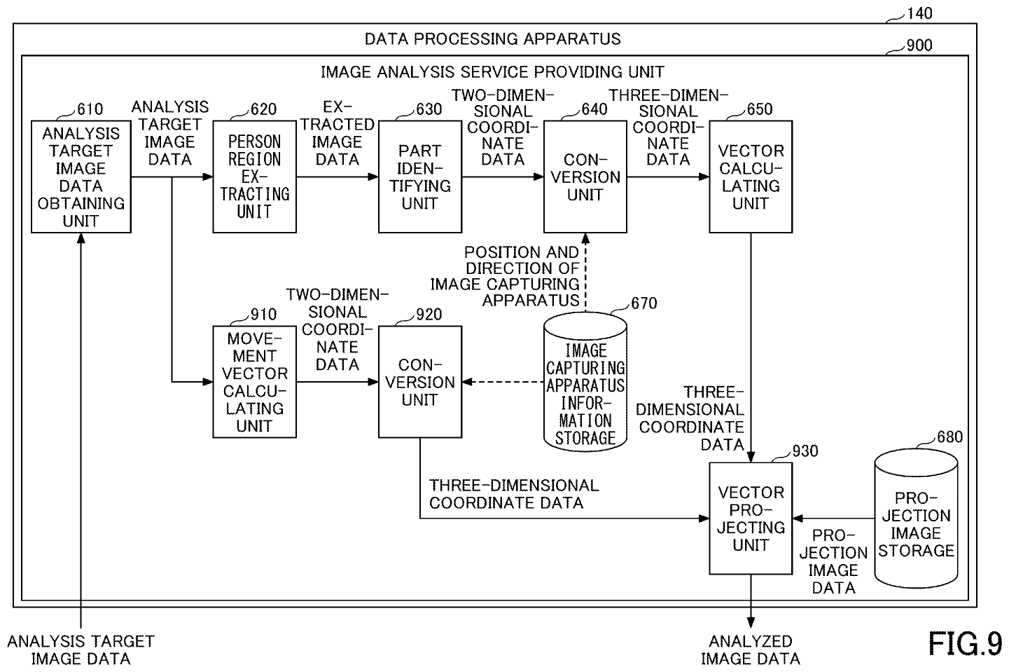

First, a functional configuration of an image analysis service providing unit will be described in detail. FIG. 9 is a second diagram illustrating an example of a functional configuration of an image analysis service providing unit. As illustrated in FIG. 9, an image analysis service providing unit 900 of the data processing apparatus 140 according to a second embodiment differs from the image analysis service providing unit 141 of the data processing apparatus 140 according to the first embodiment, in that the image analysis service providing unit 900 includes a movement vector calculating unit 910, a conversion unit 920, and a vector projecting unit 930 in place of the vector projecting unit 660. The functions of the vector projecting unit 930 differ from those of the vector projecting unit 660.

The movement vector calculating unit 910 is an example of a calculating unit. The movement vector calculating unit 910 identifies a region of a person in each frame included in analysis target image data (time series image data), and extracts image data that includes the identified region of the person. Further, the movement vector calculating unit 910 calculates the position (two-dimensional coordinate data) of the extracted image data, and compares the calculated position of the extracted image data to the position of image data extracted from the previous frame. In this manner, the movement vector calculating unit 910 can calculate a movement vector (two-dimensional coordinate data) representing the distance and direction in which the person moves for a predetermined period of time (during a single frame period).

In the present embodiment, as the position of the extracted image data, the movement vector calculating unit 910 calculates two-dimensional coordinate data of the position on the ground of the field 200. This is because when the two-dimensional coordinate data is converted into three-dimensional coordinate data, it can be assumed that Zₛ=0.

Further, the movement vector calculating unit 910 calculates movement vectors for all people included in a frame. In addition, the movement vector calculating unit 910 calculates movement vectors for all frames included in analysis target image data. Further, the movement vector calculating unit 910 provides the calculated movement vectors (two-dimensional coordinate data) to the conversion unit 920.

The conversion unit 920 is an example of a second conversion unit. The conversion unit 920 reads, from the image capturing apparatus information storage 670, the imaging position and the imaging direction of an image capturing apparatus, which has captured analysis target image data from an overhead view, in the world coordinate system. Further, the conversion unit 920 uses the read imaging position, the read imaging direction, and the like to convert a calculated movement vector (two-dimensional coordinate data of an initial point and a terminal point) into three-dimensional coordinate data in the world coordinate system. The conversion unit 920 provides the converted movement vector (three-dimensional coordinate data of the initial point and the terminal point) to the vector projecting unit 930. The conversion process for converting the movement vector (two-dimensional coordinate data of an initial point and a terminal point) in analysis target image data into three-dimensional coordinate data in the world coordinate system has been described in the first embodiment, and thus, the description thereof will not be repeated. However, in the second embodiment, when the conversion process is performed, it is assumed that Zₛ=0.

The vector projecting unit 930 obtains a vector (three-dimensional coordinate data) derived by the vector calculating unit 650 based on the shoulders, and a movement vector (three-dimensional coordinate data) converted by the conversion unit 920.

Further, the vector projecting unit 930 determines whether the movement vector (three-dimensional coordinate data) converted by the conversion unit 920 has a predetermined length (that is, determines whether the person is moving at a speed greater than or equal to a predetermined speed).

If the vector projecting unit 930 determines that the movement vector has the predetermined length (that is, the person is moving at a speed greater than or equal to the predetermined speed), the vector projecting unit 930 projects the movement vector (three-dimensional coordinate data), converted by the conversion unit 920, onto projection image data. Conversely, if the vector projecting unit 930 determines that the movement vector does not have the predetermined length (that is, the person is not moving at a speed greater than or equal to the predetermined speed), the vector projecting unit 930 projects the vector derived by the vector calculating unit 650 based on the shoulders onto projection image data.

### <Flow of Image Analysis Service Providing Process>

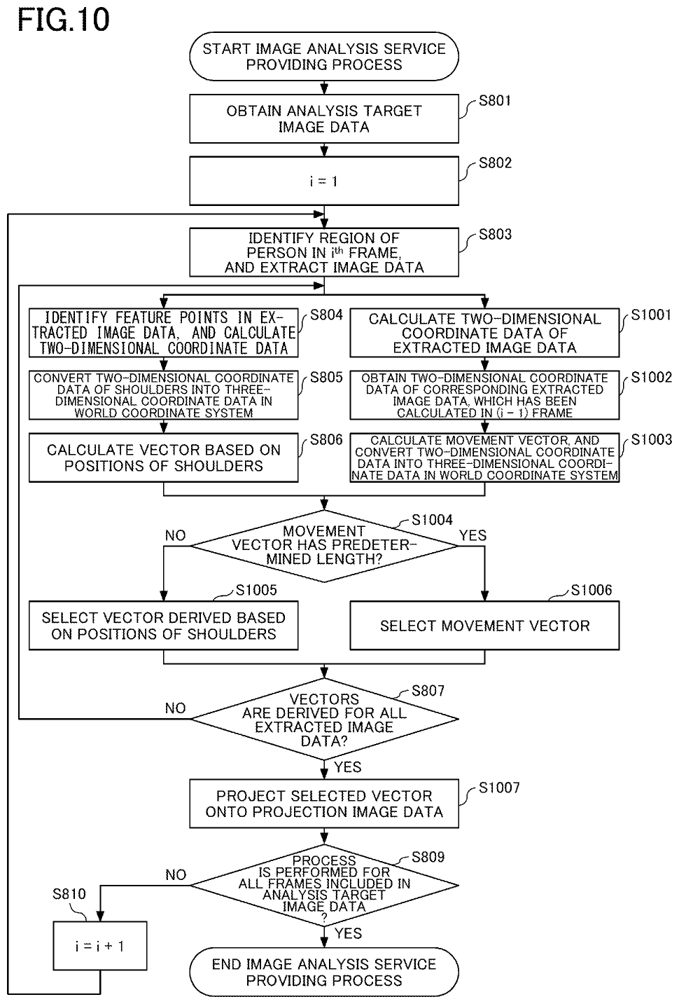

Next, a flow of an image analysis service providing process performed by the image analysis service providing unit 900 of the data processing apparatus 140 will be described. FIG. 10 is a second flowchart illustrating a flow of an image analysis service providing process. Differences from the image analysis service providing process illustrated in FIG. 8 are steps S1001 through S1006, and step S1007.

In step S1001, the movement vector calculating unit 910 identifies a region of a person in an i^{th} frame, and extracts, from the i^{th} frame, image data that includes the identified region of the person. Further, the movement vector calculating unit 910 calculates two-dimensional coordinate data of the extracted image data.

In step S1002, the movement vector calculating unit 910 obtains two-dimensional coordinate data of corresponding extracted image data, which has been calculated in a (i−1) frame.

In step S1003, the movement vector calculating unit 910 calculates a movement vector (two-dimensional coordinate data) of the identified person, based on the two-dimensional coordinate data calculated in step S1001 and the two-dimensional coordinate data obtained in step S1002. Further, the conversion unit 920 converts the calculated movement vector (two-dimensional coordinate data) into three-dimensional coordinate data in the world coordinate system.

In step S1004, the vector projecting unit 930 determines whether the movement vector converted in step S1003 has a predetermined length. If the vector projecting unit 930 determines that the movement vector does not have the predetermined length (no in step S1004), the process proceeds to step S1005.

In step S1005, the vector projecting unit 930 selects the vector derived based on the positions of the shoulders in step S806, as a vector to be projected onto projection image data.

Conversely, if the vector projecting unit 930 determines that the movement vector has the predetermined length (yes in step S1004), the process proceeds to step S1006.

In step S1006, the vector projecting unit 930 selects the movement vector converted in step S1003, as a vector to be projected onto projection image data.

In step S1007, the vector projecting unit 930 uses the three-dimensional coordinate data to project the vector, selected in step S1005 or step S1006, onto the projection image data. Further, the vector projecting unit 930 transmits the projection image data, onto which the vector has been projected, to the image data distribution apparatus 120 as analyzed image data.

### <Summary>

As is clear from the above description, in addition to the functions of the data processing apparatus according to the first embodiment, the data processing apparatus according to the second embodiment is configured to:
calculate a movement vector of a person in each frame of obtained analysis target image data, and convert the movement vector (two-dimensional coordinate data) into three-dimensional coordinate data; and
project the movement vector onto projection image data instead of a vector derived based on the positions of the shoulders, in a case where the movement vector has a predetermined length. The projection image data is a top view image associated with the world coordinates.

Accordingly, when a person is moving at a speed greater than or equal to the predetermined speed, the data processing apparatus according to the second embodiment can derive a vector representing the orientation of the body of the person based on the direction of movement (without calculating three-dimensional coordinate data of the positions of both shoulders). Therefore, according to the second embodiment, the processing load of the data processing apparatus can be reduced.

### Third Embodiment

In the image analysis service providing process according to the above-described first embodiment, the image analysis service providing process is performed for each frame and for each person; however, the image analysis service providing process is not limited thereto.

Further, in the first embodiment, a specific method for implementing the person region extracting unit 620 and the part identifying unit 630 is not mentioned. The person region extracting unit 620 and the part identifying unit 630 may be implemented by a trained model that uses an image as an input to produce an output as appropriate.

Further, in the above-described first embodiment, the data processing apparatus 140 derives a vector representing the orientation of the body of a person based on the positions of the shoulders. However, information derived by the data processing apparatus 140 based on the positions of the shoulders is not limited to a vector representing the orientation of the body of a person. The data processing apparatus 140 may derive information other than a vector representing the orientation of the body of a person.

Further, in the above-described first embodiment, the positions of the shoulders of a person are used to derive a vector representing the orientation of the body of the person. However, the position of any part other than the positions of the shoulders may be used to derive a vector representing the orientation of the body of the person. For example, in addition to the positions of the shoulders, the position of the abdomen below the shoulders may be used to calculate a vector orthogonal to a plane including three points, as a vector representing the orientation of the body of the person.

Further, in the above-described first embodiment, because the heights of the shoulders of the person are approximately the same, it is assumed that the Z coordinate is “Zₛ” in the world coordinate system. However, the heights of the shoulders of each person may be registered in a database in advance, and the database may be retained. In this case, when the data processing apparatus 140 calculates the X and Y coordinates of the positions of the shoulders of a person in the world coordinate system, the data processing apparatus 140 may identify the person and read the heights of the shoulders of the identified person from the database. Accordingly, the X and Y coordinates of the positions of the shoulders of the identified person can be calculated with high accuracy in the world coordinate system. Needless to say, the processing load of the data processing apparatus 140 can be more reduced when it is assumed that the heights of the shoulders of the person are approximately the same.

Further, in the above-described first embodiment, the positions of the shoulders of a person are calculated. However, the data processing apparatus 140 does not necessarily calculate the positions of the shoulders of a person, and may calculate the position of any other part of the person in analysis target image data. For example, the data processing apparatus 140 may be configured to calculate the positions of the left ear and the right ear of a person, the positions of the waist on the left side and the right side of a person, the positions of the left knee and the right knee of a person, and the like. If the data processing apparatus 140 calculates the positions of the ears of a person, the data processing apparatus 140 may derive a vector representing the orientation of the face of the person. If the data processing apparatus 140 calculates the positions of the waist on the left and right sides or the positions of the knees, the data processing apparatus 140 may derive a vector representing the orientation of the legs. However, in such a case, the positions in the height direction (z coordinates in the world coordinate system) of parts to be calculated are assumed to be preset in the conversion unit 640.

Further, in the above-described first embodiment, the imaging position and the imaging direction of an image capturing apparatus are fixed, and the imaging position and the imaging direction of the image capturing apparatus are read from the image capturing apparatus information storage 670.

However, if the imaging direction of the image capturing apparatus is variable, it is assumed that additional information is provided in which the imaging direction of the image capturing apparatus is associated with each frame of analysis target image data. In this case, the conversion unit 640 may be configured to read the imaging direction by referring to the additional information.

Further, in the above-described first embodiment, projection image data is a top view image viewed from above the field 200. However, projection image data is not limited to the top view image, and may be a bird's-eye view image viewed from any viewpoint.

Further, in the above-described first embodiment, the entire field 200 is included in the imaging range of a single image capturing apparatus, and the single image capturing apparatus captures an overhead image of the entire field 200. However, if a plurality of image capturing apparatuses capture overhead images such that the imaging ranges of the image capturing apparatuses include respective areas of the field 200, vectors representing the orientations of the bodies of people included in the areas of the field 200 are derived, and projected onto one projection image data. Accordingly, the orientations of all players in the field 200 can be identified.

Further, in the above-described embodiments, soccer is cited as an example of team sports. Needless to say, the above-described embodiments can be applied to any team sports other than soccer.

Further, in the above-described embodiments, an overhead image of the field is captured. However, an area whose overhead image is captured is not limited to the field. An overhead image of a place where a large crowd is gathered (an area where a large number of people are gathered) may be captured. Accordingly, the data processing apparatus 140 can perform crowd management.

Further, in the above-described embodiments, the terminal 150 receives image data and analyzed image data by requesting the image data distribution apparatus 120 to distribute the image data and the analyzed image data. However, the terminal 150 may be configured to receive image data and analyzed image data by requesting the data processing apparatus 140 to distribute the image data and the analyzed image data.

### Other Embodiments

In the above-described embodiments, the functions of each of the image analysis service providing units 141 and 900 are implemented by the processor 501 executing the image analysis service providing program. However, the functions of each of the image analysis service providing units 141 and 900 may be implemented by a circuit that is configured by an analog circuit, a digital circuit, or an analog-digital mixture circuit. In addition, a control circuit for controlling the functions of each of the image analysis service providing units 141 and 900 may be provided. Each of the circuits may be an application-specific integrated circuit (ASIC), a field-programmable gate array (FPGA), or the like.

In the above-described embodiments, when the image analysis service providing program is executed, the image analysis service providing program may be stored in a recording medium such as a flexible disk or a CD-ROM, loaded into a computer, and executed. The recording medium is not limited to a removable medium such as a magnetic disk or an optical disc, and may be a fixed-type recording medium such as a hard disk device or a memory. Further, the processes executed by the software may be implemented by a circuit such as a FPGA and may be executed by hardware.

It should be noted that the present invention is not limited to the above-described configurations, such as the configurations described in the above-described embodiments, and combinations with other elements. In these respects, various modifications can be made within the scope of the invention without departing from the spirit of the invention, and the configurations may be appropriately determined according to an application form.

What is claimed is:

1. A data processing apparatus comprising:
at least one memory; and
at least one processor;
wherein the at least one processor is configured to
obtain two-dimensional data relating to a position of a predetermined part of an object in a first image, the two-dimensional data being in a first coordinate system,
calculate three-dimensional data relating to the position of the predetermined part in a second coordinate system based on the two-dimensional data relating to the position of the predetermined part in the first coordinate system, and
obtain information indicating an orientation of the object based on the calculated three-dimensional data relating to the position of the predetermined part in the second coordinate system.

2. The data processing apparatus according to claim 1, wherein the calculating uses one or more pieces of preset data relating to the position of the predetermined part of the object, to calculate the three-dimensional data in the second coordinate system based on the two-dimensional data in the first coordinate system.

3. The data processing apparatus according to claim 1, wherein the obtaining of the two-dimensional data identifies the predetermined part of the object in the first image based on an analysis of the first image.

4. The data processing apparatus according to claim 1, wherein the at least one processor is configured to transmit the information indicating the orientation of the object to display the information on a display device.

5. The data processing apparatus according to claim 1, wherein the at least one processor is configured to project the information indicating the orientation of the object onto projection image data that corresponds to a top view image, and the projection image data onto which the information is projected is displayed on a display device.

6. The data processing apparatus according to claim 1, wherein the at least one processor is configured to use the calculated three-dimensional data relating to the position of the predetermined part in the second coordinate system to calculate a vector indicating the orientation of the object in the second coordinate system, and obtain the information indicating the orientation of the object based on the calculated vector in the second coordinate system.

7. The data processing apparatus according to claim 1, wherein the first coordinate system is an image coordinate system, and the second coordinate system is a world coordinate system.

8. The data processing apparatus according to claim 1, wherein the information indicating the orientation of the object is information indicating the orientation of the object viewed from a predetermined viewpoint.

9. The data processing apparatus according to claim 1, wherein the first image is captured by a device, and
wherein the at least one processor is configured to use the two-dimensional data relating to the position of the predetermined part in the first coordinate system, an imaging position and an imaging direction of the device, and a height of the predetermined part to calculate the three-dimensional data relating to the position of the predetermined part in the second coordinate system.

10. The data processing apparatus according to claim 9, wherein the imaging position and the imaging direction of the device, and the height of the predetermined part are represented in the second coordinate system.

11. The data processing apparatus according to claim 1, wherein the three-dimensional data relating to the position of the predetermined part in the second coordinate system is a set of three-dimensional coordinates indicating the position of the predetermined part in the second coordinate system.

12. The data processing apparatus according to claim 11, wherein the two-dimensional data relating to the position of the predetermined part in the first coordinate system is a set of two-dimensional coordinates indicating the position of the predetermined part in the first coordinate system.

13. The data processing apparatus according to claim 1, wherein the at least one processor is configured to identify a region that includes the object in the first image, identify the position of the predetermined part in the region, and obtain the two-dimensional data relating to the position of the predetermined part in the first coordinate system based on the identified position.

14. The data processing apparatus according to claim 1, wherein the first image is captured from an overhead viewpoint, and
wherein the at least one processor is configured to project the information indicating the orientation of the object onto a second image viewed from a predetermined viewpoint, so as to obtain viewpoint information indicating the orientation of the object viewed from the predetermined viewpoint, the information indicating the orientation of the object being obtained based on the three-dimensional data relating to the position of the predetermined part in the second coordinate system.

15. The data processing apparatus according to claim 1, wherein the object is a person, and
wherein the at least one processor is configured to identify a position of each of a left shoulder and a right shoulder of the person, and convert two-dimensional coordinates of the position of each of the left shoulder and the right shoulder of the person into three-dimensional coordinates in the second coordinate system.

16. The data processing apparatus according to claim 15, wherein the at least one processor is configured to calculate a first vector based on the three-dimensional coordinates of the position of each of the left shoulder and the right shoulder in the second coordinate system, and project the first vector onto a second image viewed from a predetermined viewpoint, so as to obtain a second vector representing the orientation of the object, the second vector representing the orientation of the object corresponding to the viewpoint information indicating the orientation of the object viewed from the predetermined viewpoint.

17. The data processing apparatus according to claim 16, wherein the second image viewed from the predetermined viewpoint is a top view image viewed from above a predetermined area, the predetermined area including an area captured from an overhead viewpoint.

18. The data processing apparatus according to claim 16, wherein time series images are captured by a device;
wherein the at least one processor is further configured to calculate a first movement vector of the person for a predetermined period of time based on the time series images, and convert two-dimensional coordinates of an initial point and a terminal point of the movement vector into three-dimensional coordinates in a world coordinate system based on a position and a direction of the device, and
wherein, in a case where the converted first movement vector has a predetermined length, the at least one processor projects, instead of the first vector calculated based on the position of each of the left shoulder and the right shoulder of the person, the first movement vector onto the second image viewed from the predetermined viewpoint, so as to obtain a second movement vector representing the orientation of the object, the second movement vector indicating the orientation of the object corresponding to the viewpoint information indicating the orientation of the object viewed from the predetermined viewpoint.

19. An image analysis method performed by at least one processor, the method comprising:
obtaining two-dimensional data relating to a position of a predetermined part of an object in a first image, the two-dimensional data being in a first coordinate system,
calculating three-dimensional data relating to the position of the predetermined part in a second coordinate system, based on the two-dimensional data relating to the position of the predetermined part in the first coordinate system, and
obtaining information indicating an orientation of the object based on the calculated three-dimensional data relating to the position of the predetermined part in the second coordinate system.

20. A non-transitory recording medium having stored therein a program for causing at least one processor to execute a process comprising:
obtaining two-dimensional data relating to a position of a predetermined part of an object in a first image, the two-dimensional data being in a first coordinate system,
calculating three-dimensional data relating to the position of the predetermined part in a second coordinate system, based on the two-dimensional data relating to the position of the predetermined part in the first coordinate system, and
obtaining information indicating an orientation of the object based on the calculated three-dimensional data relating to the position of the predetermined part in the second coordinate system.
